(19) United States
(12) Patent Application Publication
(10) Pub. No.: US 2024/0325589 A1
(43) Pub. Date: Oct. 3, 2024

(54) Loudspeaker with Negative Air Ions Generator

(71) Applicant: Team of Deccon Inc., Taipei City (TW)

(72) Inventors: Nai-Han Chen, Taipei City, (TW); Ting-Jung Cheng, Taipei City, (TW)

(21) Appl. No.: 18/192,825

(22) Filed: Mar. 30, 2023

(51) Int. Cl. A61L 9/22; H01T 23/00; H04R 1/02; H04R 3/00

(52) U.S. Cl. CPC A61L 9/22; H01T 23/00; H04R 1/023; H04R 1/025; H04R 1/028; H04R 3/007; A61L 2209/134; H04R 2201/021; H04R 2499/13

(57) ABSTRACT

This present invention provides a loudspeaker with negative air ions generator, wherein installed on the loudspeaker in the existing car or home in the form of an embedded, box-shaped or external box, an anion generator, a high-voltage pluggable connector and a high-voltage recharge diode board, using the anti-high voltage recharge diode board to prevent the high-voltage current generated by the negative air ions generator from recharging to other electronic devices in the car, and solve the problem of the speaker through structural design and material treatment the magnetic field of the magnet and the loudspeaker grille have the effect of eliminating negative air ions, so that the loudspeaker generates a large amount of negative air ions to cover the entire car or home to clean the air.

### BACKGROUND OF THE INVENTION

### Field of the Invention

The present invention relates to a loudspeaker with negative air ions generator, and more particularly to a device that applies negative air ions generators to automobiles and household loudspeakers to purify the air in cars and homes.

### b) Description of the Prior Art

The negative air ions generator used in general automobiles is mainly to insert the negative air ions emitter into the socket of the car cigarette lighter after being made into a cylinder. However, the above negative air ions generator can only be around 30 centimeters around the cigarette lighter. It will produce the cleaning effect of negative air ions. It is not close enough to passengers, and it is difficult to clean the entire space in the car. Moreover, this product will also occupy the use space of the car cigarette lighter. Generally, household negative air ions generators are installed in the cleaning machine. The cleaning machine is quite large and takes up a lot of space, and the sound of the fan is noisy. negative air ions can only be effective around the cleaning machine, and usually not It will be near the user, so there is room for improvement.

In view of this, how to make all the spaces in the car clean, so that every passenger in the car has its own exclusive air purifier, and how to make the home environment closer to the user, or use a negative air ions emitter There are many ways to greatly expand the generation and coverage of negative air ions to achieve no dead angle and full coverage, so that they can exert a greater cleaning effect, which has become the problem that this creation wants to solve.

The author has accumulated many years of experience, carefully researched and continuously tested and improved, and now he has finally created an innovative speaker with the function of generating negative air ions, which can eliminate the shortcomings of conventional products and improve the efficacy.

### SUMMARY OF THE INVENTION

The primary object of the present invention is to provide a loudspeaker with negative air ions generator, which is equipped with a negative air ions generator on the sides of the four doors of the car or other positions in the car and on the speakers used at home, so that the car and the amount and coverage of negative air ions at home are far greater than those of conventional negative air ions generators that are only plugged into the cigarette lighter or installed on the cleaner.

In order to achieve the above purpose, the present invention adopts the following technical means:

A speaker with the function of generating negative air ions. A negative air ions generator is installed on the side of the car door or other places in the car or on the home speaker, a high-voltage resistant pluggable connector (only required for non-box type car speaker) and high-voltage recharge diode board, wherein the negative air ions generator is electrically connected with the speaker, the negative air ions high-voltage carbon brush head of the negative air ions generator penetrates the center of the speaker through the anti-high voltage pluggable connector and connects to the negative air ions generator body attached to the speaker (Only the non-box type car speaker needs to be penetrated), the anti-high voltage recharge diode board is installed near the negative air ions generator, and the anti-high voltage recharge diode board can prevent the high voltage and current generated from being fed back into other electronic devices, and the influence of the magnetic field of the speaker magnet and the negative air ions elimination effect of the speaker grille can be solved through structural design and material treatment.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a structural schematic view of the back of the present invention.

FIG. 2 shows a structural schematic view of the front of the present invention.

FIG. 3 shows a structural schematic view of the present invention.

FIG. 4 shows a structural schematic view of assembly of the back of the present invention.

FIG. 5 shows a structural view of assembly of the front of the present invention.

FIG. 6 shows a schematic view of embodiment of the present invention.

FIG. 7 shows a partial structural schematic view of embodiment of the present invention.

FIG. 8 shows a structural schematic view of the back of the home ceiling of the present invention.

FIG. 9 shows a structural schematic view of the front of the home ceiling of the present invention.

FIG. 10 shows a structural schematic view of the back of the desktop loudspeaker of the present invention.

FIG. 11 shows a structural schematic view of the front of the desktop loudspeaker of the present invention.

### DETAILED DESCRIPTION OF THE PREFERRED EMBODIMENTS

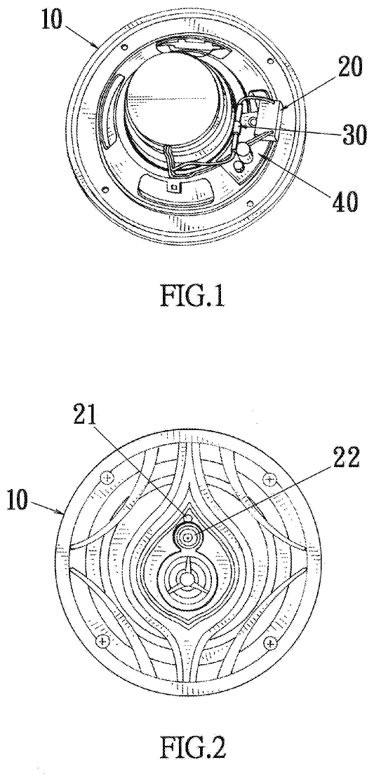

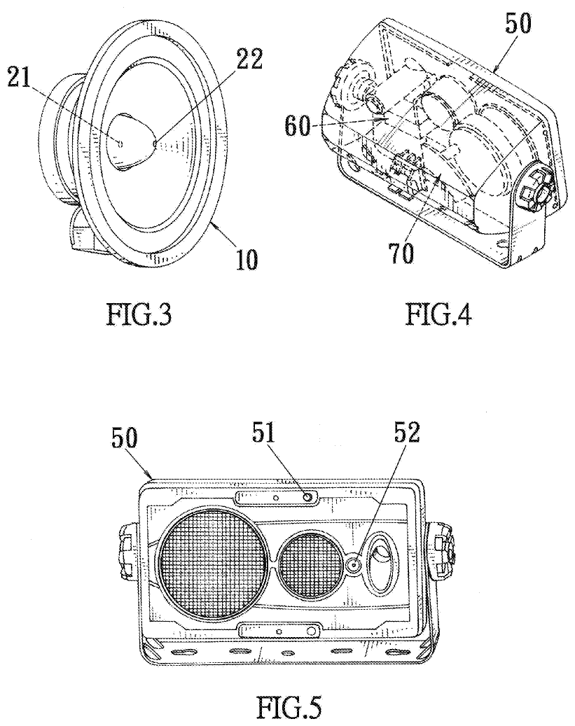

Referring to FIG. 1 show loudspeaker with negative air ions generator according to a first embodiment of the present invention, the loudspeaker 10 configured on the door side is equipped with a negative air ions generator 20, an anti-high voltage pluggable connector 30 and an anti-high voltage recharge diode board 40, wherein said negative air ions generator 20 is used to penetrate said loudspeaker 10 to the front net through the anti-high voltage pluggable connector 30, and said anti-high voltage recharge diode board 40 is installed near said negative air ions generator 20, said anti-high voltage recharge diode board 40 can prevent the high-voltage recharge to other electronic devices in the car when negative air ions are generated. Structural design and material selection are used to resolve the elimination effect caused by the magnetic field and speaker grille material on negative air ion generation. Also referring to FIG. 2, wherein said loudspeaker 10 is equipped with a negative air ions working indicator light 21 and a negative air ions high pressure carbon brush head 22, when said negative air ions high pressure carbon brush head 22 discharges, the air forms negative air ions through its negative electric shock, at this time said negative air ions working indicator light 21 presents a lighting state; and as shown in FIG. 3, said negative air ions working indicator light 21 and said negative air ions high pressure carbon brush head 22 can be located at the same place.

Referring to FIG. 4 shows loudspeaker with negative air ions generator according to a second embodiment of the present invention, a negative air ions generator 60 and an anti-high voltage recharge diode board 70 are installed on the loudspeaker 50, wherein said negative air ions generate 60 is electrically connected with the loudspeaker 50, and said anti-high voltage recharge diode board 70 is installed on the side of the negative air ions generator 60, said anti-high voltage recharge diode board 70 can prevent the high voltage recharge generated together when negative air ions are generated irrigate to other electronic equipment in the car; Referring to FIG. 5, wherein said loudspeaker 50 is installed with a negative air ions working indicator light 51 and a negative air ions high pressure carbon brush head 52.

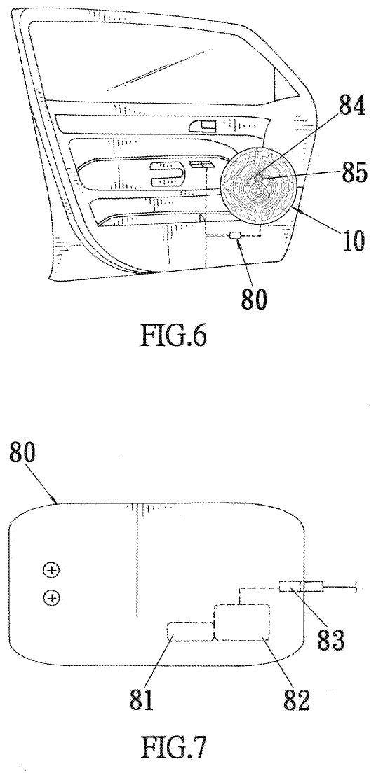

Referring to FIG. 6 and FIG. 7 shows loudspeaker with negative air ions generator according to a third embodiment of the present invention, wherein a loudspeaker 10 configured on the side of the car door is electrically connected to a negative air ions generator external box 80, and said negative air ions general external box 80 includes an anti-high voltage recharge diode board 81, a negative air ions generator 82 and an anti-high voltage pluggable connector 83, wherein said negative air ions generator 82 connects with said anti-high voltage pluggable connector 83 with said high-voltage recharge board 81 are electrically connected; a negative air ions working indicator light 84 and a negative air ions high pressure carbon brush head 85 are installed on said loudspeaker 10, and said anti-high voltage recharge diode board 81 can prevent the high-voltage current generated together with negative air ions from being fed back to other electronic devices in the car; if consumers want to increase the magnet size behind the negative air ions speaker 10 to increase the power supply wattage and the sound pressure of the loudspeaker, said negative air ions generator 82 must use said negative air ions generator external box 80 to complete in an external manner.

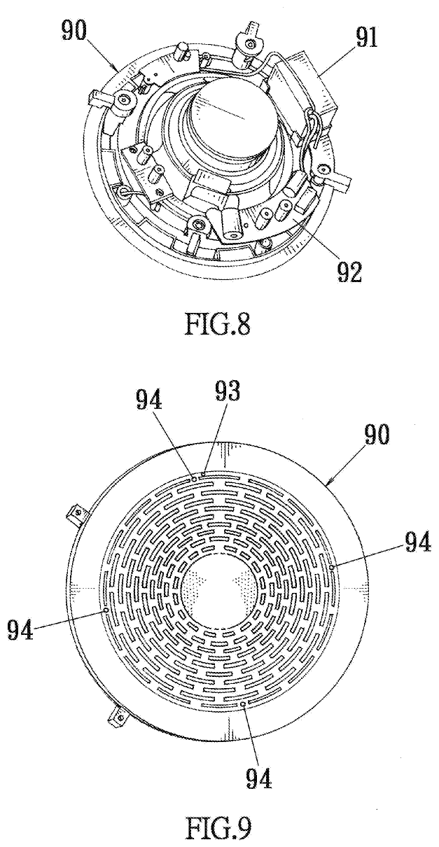

Referring to FIG. 8 shows ceiling loudspeaker with negative air ions generator according to a fourth embodiment of the present invention, wherein a ceiling loudspeaker 90 is to install a reinforced negative air ions generator 91 and an anti-high voltage recharge diode board 92; as shown in FIG. 9, wherein said ceiling loudspeaker 90 is equipped with a negative air ions working indicator light 93 and four negative air ions high pressure carbon brush heads 94. Said anti-high voltage recharge diode board 92 can prevent the high-voltage current generated together from being recharged to other electronic devices when negative air ions are generated; this type of enhanced negative air ions generator 91 is a one-in four-out type, and four high negative air ions high pressure carbon brush heads 94 are distributed in four different directions on the front of said speaker 90, which not only increases the production of negative air ions by four times, but also four different directional injection can achieve 360-degree full coverage to effectively clean indoor air.

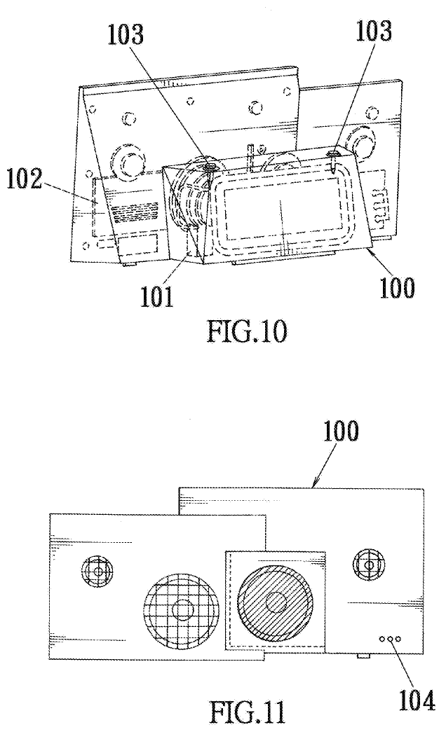

Referring to FIG. 10 shows loudspeaker with negative air ions generator according to a fifth embodiment of the present invention, wherein a loudspeaker 100 is equipped with a reinforced negative air ions generator 101, an anti-high voltage recharge diode board 102 and two negative air ions high pressure carbon brush heads 103; as shown in FIG. 11, wherein said loudspeaker 100 is equipped with a negative air ions working indicator light 104, and said anti-high voltage recharge diode board 102 integrated into the motherboard can prevent the high-voltage current generated together with negative air ions from being fed back to other electronic devices; this type of enhanced negative air ions generator 101 is a one-in two-out type, and two negative air ions high pressure carbon brush heads 103 are distributed in two different directions above the back of the loudspeaker 100, which not only doubles the size the amount of negative air ions generated, and one left and one right two different directions can achieve a larger area coverage, said kind of desktop loudspeaker 100 is very close to the user, which can effectively clean the air around the user.

In light of above, and in accordance with the above description of the embodiments, the present invention has a speaker that produces negative air ions, which can be equipped with an air purifier with a negative air ions generator on the four doors of the car or other positions in the car and speakers used at home to make negative air ions, the amount covers the entire car or home space, and is closer to passengers or home users to purify the air. Its practical effect is beyond doubt, and this creation has never been published in the world or has been seen in other publications, and it is in compliance with the provisions of the patent law, and a patent application is filed in accordance with the law.

It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. A loudspeaker with negative air ions generator, wherein a loudspeaker is equipped with a negative air ions generator, a high-voltage resistant pluggable connector and a high-voltage rechargeable diode board, wherein said negative air ions generator is electrically connected to the loudspeaker, said negative air ions generator is used to penetrate the loudspeaker to the front grille through the anti-high voltage pluggable connector, said anti-high voltage recharge diode board is installed near the negative air ions generator, said anti-high voltage recharge diode board can prevent the high-voltage current generated together with negative air ions from being fed back to other electronic devices in the car, and the influence of the magnetic field of the loudspeaker magnet and the negative air ions elimination effect of the loudspeaker grille material on the negative air ions is solved through structural design and material treatment.

2. The loudspeaker with negative air ions generator according to claim 1, wherein said negative air ions working indicator light and a negative air ions high pressure carbon brush head are installed on the loudspeaker.

3. A loudspeaker with negative air ions generator, wherein a loudspeaker arranged on the side of the car door is electrically connected to an external box of the negative air ions generator, said external box of the negative air ions generator includes an anti-high voltage recharge diode board, an anti-high voltage pluggable connector, wherein said negative air ions generator is electrically connected to the speaker through the anti-high voltage pluggable connector.

4. The loudspeaker with negative air ions generator according to claim 3, wherein said negative air ions working indicator light and a negative air ions high pressure carbon brush head are installed on the loudspeaker.

5. A ceiling loudspeaker with negative air ions generator, wherein a ceiling loudspeaker is equipped with a negative air ions generator, negative air ions high pressure carbon brush head.

6. The ceiling loudspeaker with negative air ions generator according to claim 5, wherein said negative air ions generator on the ceiling loudspeaker is released by four negative air ions high pressure carbon brush heads, which is a one-in four-out type negative air ions generator.

7. A loudspeaker with negative air ions generator, wherein a negative air ions generator is installed on the home desktop speaker, said negative air ions generator is electrically connected to an anti-high voltage recharge diode plate and two negative air ions high pressure carbon brush heads.

8. The loudspeaker with negative air ions generator according to claim 7, wherein said negative air ions working indicator light is installed on the loudspeaker, and said negative air ions generator is released by two negative air ions high pressure carbon brush heads of negative air ions, which is a type of one-in two-out of the negative air ions generator.
